(19) United States
(12) Patent Application Publication
(10) Pub. No.: US 2009/0081266 A1
(43) Pub. Date: Mar. 26, 2009

(54) SKINCARE MASK PACKET

(76) Inventors: Nai-Ko Ying, Taipei City (TW); Jeng-Horng Hou, Taipei City (TW)

Correspondence Address: HDLS Patent & Trademark Services, P.O. BOX 220746, CHANTILLY, VA, 20153-0746, US

(21) Appl. No.: 12/056,411

(22) Filed: Mar. 27, 2008

(30) Foreign Application Priority Data
May 18, 2007 (TW) 096208057

(51) Int. Cl. A61K 8/02

(52) U.S. Cl. 424/402

(57) ABSTRACT

A skincare mask packet includes a packing bag, a skincare mask, and a skincare liquid packaging unit. An outer edge of the packing bag has a sealing function to form a packet configuration. In addition, the skincare mask is made of materials capable of absorbing liquid. An appropriate amount of skincare liquid is further enclosed in the skincare liquid packaging unit that is accommodated in the packing bag together with the skincare mask. During usage, the packing bag is first pressed or squeezed to break the skincare liquid packaging unit, making the skincare liquid flow out the unit and absorbed by the skincare mask. After the skincare liquid has penetrated into the skincare mask, the packing bag may be sealed off for taking out the skincare mask for a skin care.

### BACKGROUND OF THE INVENTION

1. Field of the Invention

The present invention generally relates to a skincare product, in particular, to a bag packet of skincare mask, for example, hand mask, foot mask and face mask.

2. Description of Prior Art

Accordingly, common skincare mask product, for example, hand mask, foot mask, and face mask, etc., is enclosed in a bag packet made of aluminum foil or plastic, after absorbing an appropriate amount of skincare liquid. In the meantime, another appropriate amount of skincare liquid is also contained in the bag packet to keep the skincare mask with certain wetness. When a user is intended to skin care, he only has to tear the bag packet, takes out the skincare mask, and covers it on human parts, for example, face, hand, and foot, etc., for reaching a skincare effectiveness.

However, since a skincare mask has to be mixed with skincare liquid before it is enclosed in a bag packet to be sealed during a packing operation according to the prior arts, the skincare liquid is vulnerable to deterioration due to its contact with ambient air before being packed. Therefore, the preservation time of this kind of skincare mask product is very short. In particular, after the skincare liquid being deteriorated, the product not only has no skincare effectiveness, but also may further hurt user's skin, and a medical care may be further needed if the skin is seriously hurt.

Accordingly, aiming to solve aforementioned shortcomings, after a substantially devoted study, in cooperation with the application of relatively academic principles, the inventor finally proposes the present invention that is designed reasonably to possess the capability of improving the prior arts significantly.

### SUMMARY OF THE INVENTION

The main objective of the invention is to provide a bag packet of skincare mask, wherein an appropriate amount of skincare liquid is packaged in a skincare liquid packaging unit, which is then enclosed in a bag packet together with the skincare mask. When a user is intended to skin care, he breaks the skincare liquid packaging unit to mix the skincare liquid into the skincare mask. In this manner, it is possible to keep the skincare liquid from contacting with the ambient air after the packaging operation and before the product usage, so the preservation time of the product may be prolonged, ensuring the skincare liquid without the vulnerability of deterioration.

To achieve aforementioned objective, the invention is to provide a skincare mask packet that includes a packing bag, a skincare mask, and a skincare liquid packaging unit. Wherein, the outer edge of the packing bag has a sealing function to form a packet configuration. In addition, the skincare mask is made of materials capable of absorbing liquid. An appropriate amount of skincare liquid is further enclosed in the skincare liquid packaging unit that is accommodated in the packing bag together with the skincare mask. During usage, the packing bag is first pressed or squeezed to break the skincare liquid packaging unit, making the skincare liquid flow out of the unit and absorbed by the skincare mask. After the skincare liquid has penetrated into the skincare mask, the packing bag may be sealed off for taking out the skincare mask for a skin care. Since the skincare liquid is preserved in the skincare liquid packaging unit until the product usage, it is possible to keep the skincare liquid form deterioration for a long time to thereby achieve aforementioned objective.

### BRIEF DESCRIPTION OF DRAWING

The features of the invention believed to be novel are set forth with particularity in the appended claims. The invention itself, however, may be best understood by reference to the following detailed description of the invention, which describes a number of exemplary embodiments of the invention, taken in conjunction with the accompanying drawings, in which:

FIG. 1 is an explosive illustration of the invention applied in a hand mask;

FIG. 2 is an assembled illustration of the invention applied in a hand mask;

FIG. 3 is an assembled illustration of the invention applied in a foot mask;

FIG. 4 is a plane illustration of the invention, wherein a face mask is fully developed;

FIG. 5 is a sectional illustration of the invention applied in an unsealed off face mask; and

FIG. 6 is a sectional illustration of the invention applied in a sealed off face mask.

### DETAILED DESCRIPTION OF THE INVENTION

In cooperation with attached drawings, the technical contents and detailed description of the present invention are described thereinafter according to a number of preferable embodiments, being not used to limit its executing scope. Any equivalent variation and modification made according to appended claims is all covered by the claims claimed by the present invention.

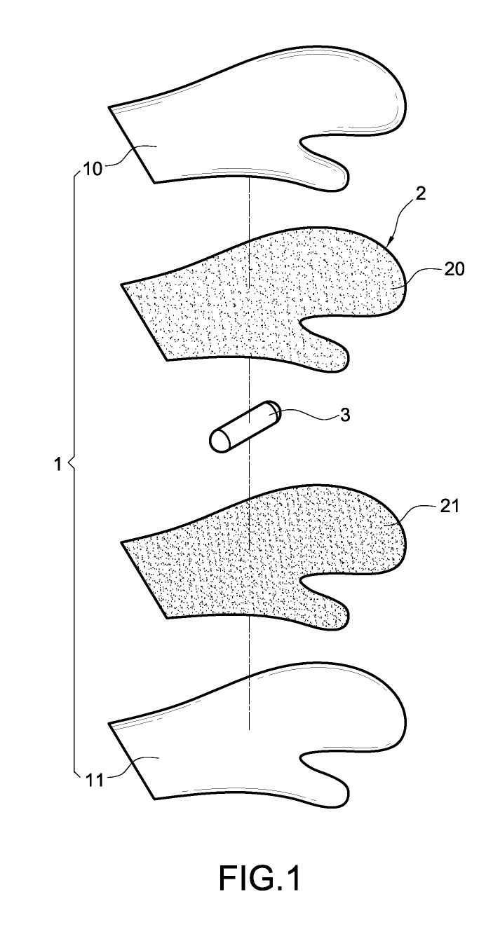

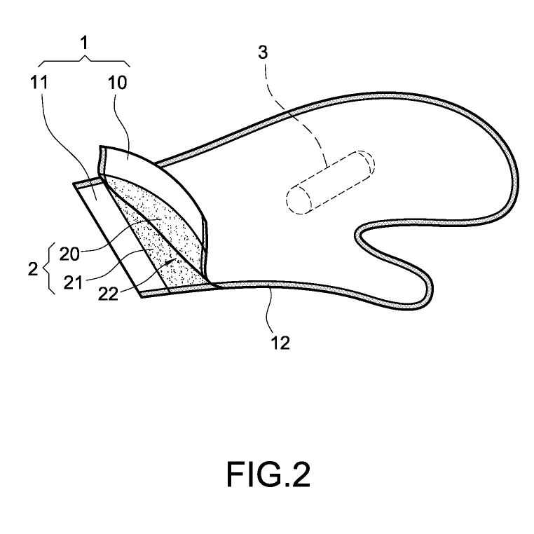

Please refer to FIG. 1 and FIG. 2, which respectively are explosive illustration and assembled illustration according to the present invention applied in a hand mask. The present invention is to provide a skincare mask packet, including a packing bag 1, a skincare mask 2, and a skincare liquid packaging unit 3.

Wherein, the packing bag 1 may be made of materials of aluminum foil or plastic to enclose the skincare mask 2 and the skincare liquid packaging unit 3 for constituting a bag packet, which comprises a first envelope 10 and a second envelope 11, both of which are overlapped with their outer edges sealed to form a sealing edge 12 at the outer edge of the packing bag 1, as shown in FIG. 2.

The skincare mask 2 is mainly made of materials, for example, nonwoven, textile, or air laid paper, etc., capable of absorbing liquid. Before a product usage, the skincare mask 2 will not absorb any skincare liquid. In this case, since the skincare mask 2 is applied for a hand skin care, the profiles of the skincare mask 2 and the packing bag 1 may be made in corresponding to a profile of human palm. Furthermore, the skincare mask 2, accommodated in the packing bag 1 and laid between the first and second envelopes 10, 11, comprises a first and second linings 20, 21, both of which are overlapped and sealed together with the sealing edge 12 of the packing bag 12, except for reserving an opening 22 for a human palm to pass through. Thereby, a human hand may be fitted into the skincare mask 2.

The skincare liquid packaging unit 3, containing an appropriate amount of skincare liquid (i.e., the so-called “serum” for skin care or repair), is accommodated in the packing bag 1 or further positioned between the first and second linings 20, 21. In this case, the skincare liquid packaging unit 3 is a capsule, by the configuration of which the skincare liquid is packaged. Thereby, since the skincare liquid is packaged in the skincare liquid packaging unit 3 simultaneously with the packaging operation of the packing bag 1, the skincare liquid won't contact with the ambient air directly. In the meantime, because the skincare liquid is protected from contacting with the ambient air by the capsule configuration of the skincare liquid packaging unit 3, the preservation time of the skincare liquid may be prolonged to avoid the vulnerability of deterioration, which is caused by an inadequate influence of longtime contact with ambient air.

However, when a user is intended to skin care, he only has to press and break the skincare liquid packaging unit 3 configured as a capsule, making the skincare liquid flow out the skincare liquid packaging unit 3 and absorbed by the skincare mask 2, before the packing bag 1 is sealed off. After the skincare liquid is uniformly mixed up and penetrated thoroughly into each part of the skincare mask 2, the packing bag 1 may then be sealed off to pass the human palm into the opening 22 thereof, thereby an effectiveness of skin care being able to be achieved.

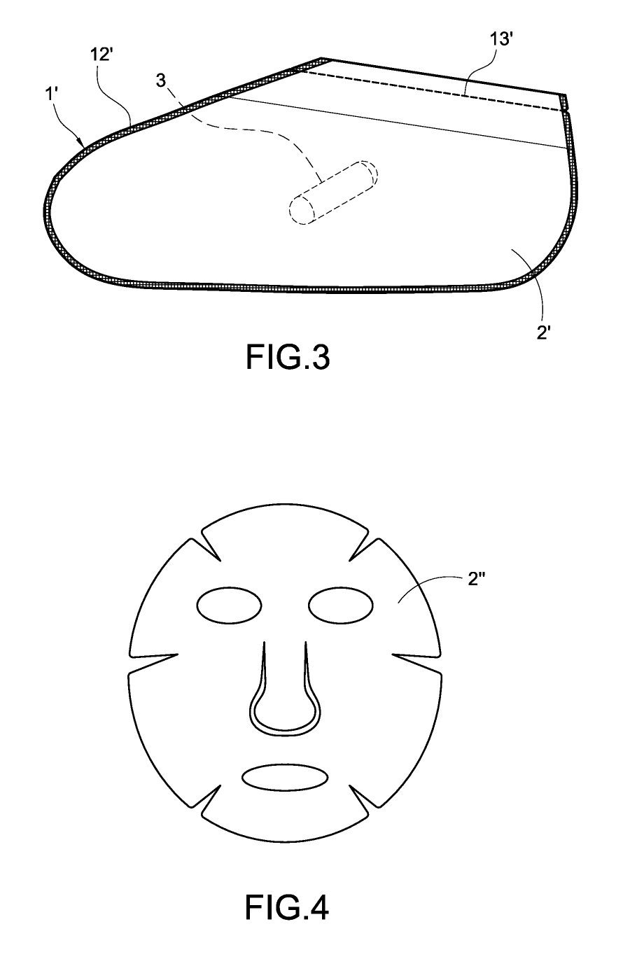

In addition, as shown in FIG. 3, it is an assembled illustration according to the invention applied as a foot mask. When the invention is applied as a foot mask, a packing bag 1′ and a skincare mask 2′ may be overlapped to be configured as a profile of a human foot and sealed at the outer edges thereof to form a sealing edge 12′. However, a sealing off line 13′ is formed at the upper part of the foot configuration for sealing off the packing bag 1 during the usage of skincare mask 2′. In this case, the skincare liquid packaging unit 3′ is still kept as a capsule configuration.

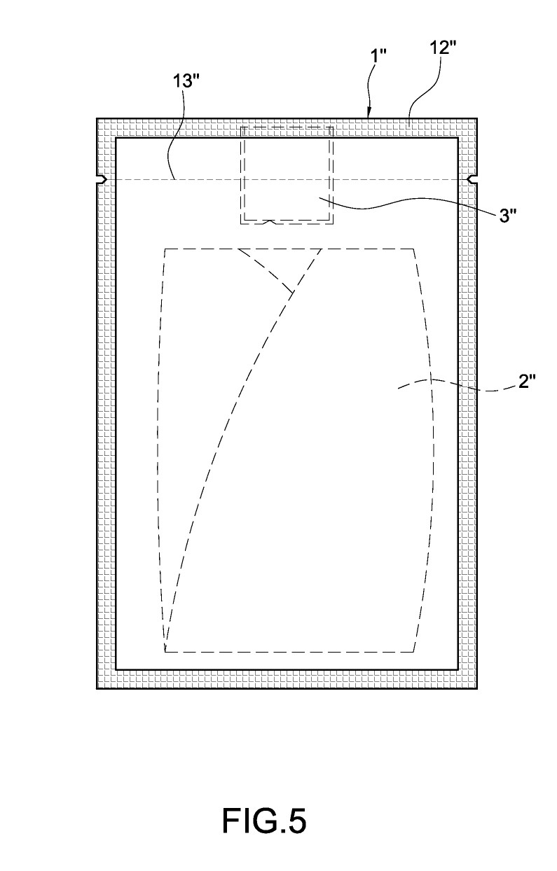

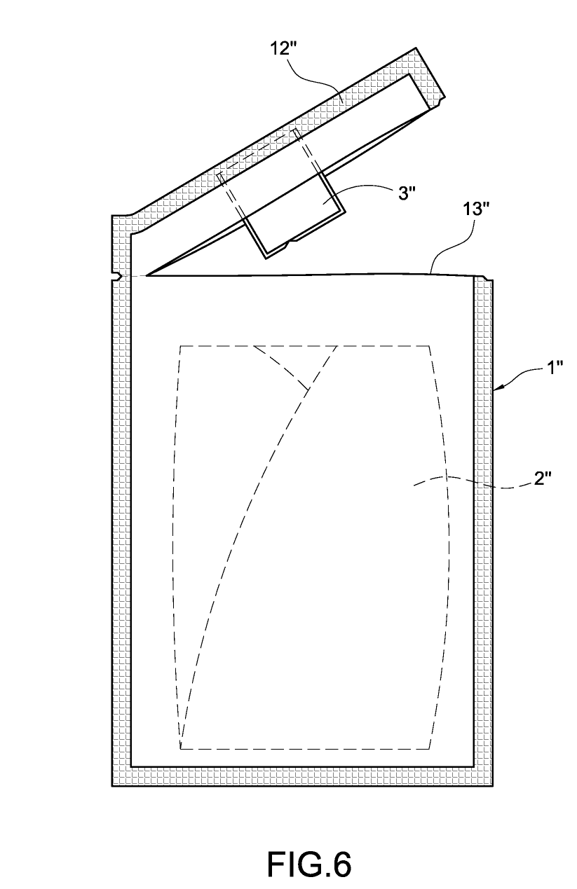

Furthermore, as shown in FIG. 4, when the invention is applied as a face mask, a skincare mask 2″ is also configured as a profile of a human face. As shown in FIG. 5, the skincare mask 2″ is folded up in a packing bag 1″, along the outer edge of which the product is closely sealed by a sealing edge 12″. In this case, the skincare liquid is packaged in a skincare liquid packaging unit 3″ by means of a thin plastic film. It may be possible to make the skincare liquid packaging unit 3″ further positioned at a sealing off line 13″ of the packing bag 1″, which may be applied as a tearing position, when the packing bag 1″ is sealed off, as shown in FIG. 6. Therefore, after the skincare liquid packaging unit 3″ being squeezed and broken to make the skincare liquid originally packaged therein flow into the skincare mask 2″, a user may take out the skincare mask 2″ by only tearing the packing bag 1″ along the sealing off line 13″, leaving the skincare liquid packaging unit 3″ and the empty plastic film in the packing bag 1″, all of which may be discarded after the wetted skincare mask 2″ having been taken out, so it is possible to further enhance the applying convenience of the present invention.

Therefore, it is possible to obtain a skincare mask packet according to the present invention, by means of the structural assembly mentioned thereinbefore.

Thanks to the skincare mask packet according to the present invention, which may package the skincare mask and the skincare liquid packaging unit in a way, such that the skincare liquid is packaged in the skincare liquid packaging unit without contacting with ambient air after the packaging operation and before a user's skin care, the preserving time of the skincare product thereby being able to be prolonged, keeping the skincare liquid from the vulnerability of deterioration.

Summarizing aforementioned description, the invention is an indispensable product of novelty indeed, which may positively reach the expected usage objective for solving the drawbacks of the prior arts, and which extremely possesses the innovation and progressiveness for completely fulfilling the applying merits of new type patent, according to which the invention is applied. Please examine the application carefully and grant it as a formal patent for protecting the rights of the inventor.

However, the aforementioned description is only preferable embodiments according to the present invention, not used to limit the patent scope of the invention, so equivalently structural variation made to the contents of the present invention, for example, description and drawings, is all covered by the claims claimed thereinafter.

What is claimed is:

1. A skincare mask packet, comprising:
a packing bag, an outer edge of which has a sealing function to form a packet configuration;
a skincare mask, made of materials capable of absorbing liquid, and accommodated in the packing bag; and
a skincare liquid packaging unit, within which an appropriate amount of skincare liquid is packaged, and which is accommodated in the packing bag;
thereby, the packing bag being first pressed or squeezed to break the skincare liquid packaging unit, making the skincare liquid flow out of the unit and absorbed by the skincare mask, the packing bag then being able to be sealed off for taking out the skincare mask for a skin care, after the skincare liquid having penetrated into the skincare mask.

2. The skincare mask packet according to claim 1, wherein a sealing off line is arranged on the packing bag.

3. The skincare mask packet according to claim 2, wherein the skincare liquid is packaged in the skincare liquid packaging unit, made of plastic film, and further positioned at the sealing off line of the packing bag.

4. The skincare mask packet according to claim 1, wherein the skincare mask, configured as a profile of a human face, is folded up and accommodated in the packing bag.

5. The skincare mask packet according to claim 1, wherein the skincare mask, configured as a profile of a human palm, contains a first lining and a second lining, both of which are overlapped and sealed together with the sealing edge of the packing bag, and the skincare mask is reserved an opening for a human palm to pass through.

6. The skincare mask packet according to claim 1, wherein the skincare mask, configured as a profile of a human foot, contains a first lining and a second lining, both of which are overlapped and sealed together with the sealing edge of the packing bag, and the skincare mask is reserved an opening for a human foot to pass through.

7. The skincare mask packet according to claim 5 or 6, wherein the skincare liquid packaging unit is further positioned between the first lining and the second lining of the skincare mask.

8. The skincare mask packet according to claim 1, wherein the skincare liquid packaging unit is a capsule, in which the skincare liquid is packaged.

9. The skincare mask packet according to claim 1, wherein the skincare liquid is packaged in the skincare liquid packaging unit made of plastic film.
